(19) United States
(12) Patent Application Publication
(10) Pub. No.: US 2024/0210310 A1
(43) Pub. Date: Jun. 27, 2024

(54) PORTABLE HYPERSPECTRAL SYSTEM

(71) Applicant: KULIA LABS, INC., San Gabriel, CA (US)

(72) Inventor: Francesco CUTRALE, San Gabriel, CA (US)

(73) Assignee: KULIA LABS, INC., San Gabriel, CA (US)

(21) Appl. No.: 18/553,730

(22) PCT Filed: Apr. 2, 2021
(86) PCT No.: PCT/US2021/025660

§ 371 Date: Oct. 2, 2023

Related U.S. Application Data
(60) Provisional application No. 63/169,075, filed on Mar. 31, 2021.

(51) Int. Cl. G01N 21/25; A61B 1/04; A61B 1/06; G01N 21/01; G01N 21/27

(52) U.S. Cl. CPC G01N 21/255; A61B 1/04; A61B 1/041; A61B 1/0638; A61B 1/0684; G01N 21/27; G01N 2021/0112; G01N 2201/0627; G01N 2201/126

(57) ABSTRACT

A capsule hyperspectral system can include: an imaging capsule having an illumination system having a plurality of light emitters configured for emitting a plurality of different lighting illuminations from the imaging capsule and a hyperspectral imaging system having at least one imaging sensor, wherein the illumination system and hyperspectral imaging system are cooperatively configured to illuminate a target with a sequence of different lighting illuminations and image the target during each of the different lighting illuminations in the sequence, and a hyperspectral processing system having at least one processor, wherein the hyperspectral processing system is operably coupled with the hyperspectral imaging system and configured to receive images of the target therefrom and generate a multispectral reflectance data cube of the target from the received images of the target.

### BACKGROUND

### Field

The present disclosure relates to a portable hyperspectral system. This disclosure also relates to a capsule hyperspectral system. This disclosure also relates to a capsule hyperspectral system with a tethered imaging capsule and a hyperspectral imaging system.

### Description of Related Art

Previously, inspection, characterization and classification of objects, samples or features has been performed in a variety of fields. Frequently, this action can be performed through imaging of the target, utilizing a color camera with red, green and blue (RGB) channels, in a human-eye visible spectral range. Multiple applications may require distinction of features with RGB values too similar. Hyperspectral imaging systems extend the imaging to a number of spectral channels, beyond the RGB limits. This added dimension extends capabilities of inspecting, characterizing and classifying objects.

A typical hyperspectral imaging system requires costly imaging devices (e.g., often starting from US $40,000 in 2020), typically with larger footprint (e.g., starting from 1 cubic inch) combined with lengthy analysis (e.g., minutes per image) and large computational capabilities. Such systems may have high power, long processing time and significant space requirements. On the other hand, a low-power device, enabling for a portable, real-time operation may increase the range of applications to a multitude of applications.

Some exemplary use cases for a hyperspectral imaging system include the following. Hyperspectral imaging systems can be used in performing agricultural inspections, which may evaluate health, maturation or quality of products on site. Landscape mapping and survey devices that use hyperspectral imaging systems may be equipped on unmanned air vehicles for real time hyperspectral assessment. Hyperspectral imaging systems configured for environmental monitoring may be mounted on a static assembly for continuous low-energy analysis. In order to study public and health safety, a hyperspectral imaging system may be assembled on automated or manual screening devices. Forensics may use a hyperspectral imaging system to detect authenticity of items such as banknotes. Robotics uses hyperspectral imaging systems to increase accuracy in automated operations requiring vision. Autonomous vehicles may improve detection of roads, streets, objects with closely-matching colors by using hyperspectral imaging systems. Heads-up displays may enable enhanced high speed, high sensitivity vision in low-light conditions with hyperspectral imaging. Medical diagnostics can use hyperspectral imaging systems to detect early stages of disease, improving patient outcome.

The use of hyperspectral imaging systems for detection of cancer could have significant impacts on health care. For example, eastern Asia with Esophageal cancer (EC) incidence rates (per 100,000) of 17.9 for males and 6.8 for females may be the biggest market that may utilize hyperspectral imaging systems for cancer detection. Esophageal screening has already been tested in China and has shown a 47% reduction in mortality risk rate for patients who were screened versus those who were not. It is thought that a less invasive, rapid, and inexpensive screening device may increase the likelihood of early detection, resulting in better patient-outcomes and lower cost for the providers.

As an example of an esophageal disease, EC often remains asymptomatic until late in the disease course, emphasizing the importance of efficient screening procedures. Endoscopy is not often recommended by physicians unless individuals are at high risk or symptomatic. The most common screening procedure used today is an upper gastro-intestinal (GI) endoscopy. The procedure involves inserting a thick, flexible tube containing a camera and light down the patient's throat. An FDA approved endoscope costs approximately US $40,000 (in 2015) with an additional US $25,000 for the image processor unit. Doctors are then able to examine the lining of the esophagus and evaluate whether further testing is needed. Due to the endoscope's large and invasive nature, deep sedation is required during the entirety of the screening, and thus makes this procedure inconvenient for screening. In part, this is because deep sedation carries a risk of respiratory failure, requiring pre and post procedural work-up and observation by trained personnel. This procedure is thus expensive in terms of equipment costs and the required sedation procedure.

Additionally, an exemplary low-resolution hardware is disclosed in the PCT application WO/2015/157727, wherein contents of this disclosure is incorporated herein in their entirety.

In view of the foregoing, it may be advantageous to have hyperspectral imaging systems of this disclosure that may provide inexpensive and efficient methods for the diagnosis of esophageal and/or liver diseases as well as other uses.

### SUMMARY

In some embodiments, the present disclosure relates to an endoscopy system. In some aspects, the endoscopy system relates to a capsule hyperspectral system. In some aspects, a capsule hyperspectral system can include a tethered imaging capsule and a hyperspectral imaging system.

In some embodiments, a capsule hyperspectral system can include: an imaging capsule having an illumination system having a plurality of light emitters configured for emitting a plurality of different lighting illuminations from the imaging capsule and a hyperspectral imaging system having at least one imaging sensor, wherein the illumination system and hyperspectral imaging system are cooperatively configured to illuminate a target with a sequence of different lighting illuminations and image the target during each of the different lighting illuminations in the sequence, and a hyperspectral processing system having at least one processor, wherein the hyperspectral processing system is operably coupled with the hyperspectral imaging system and configured to receive images of the target therefrom and generate a multispectral reflectance data cube of the target from the received images of the target. In some aspects, a tether has a capsule end coupled to the imaging capsule and a system end coupled to the hyperspectral processing system. The tether can be communicatively coupled with the hyperspectral imaging system and hyperspectral processing system so as to pass data therebetween. In some aspects, the illumination system comprises at least three LEDs having at least three different color bands, such as at least one LED is a white light LED, and/or at least two LEDs are colored LEDs with different color bands. This can include an uniformly arranged array of a plurality of LEDs, such as at least six LEDs that include at least two white light LEDs and at least four colored LEDs with at least two different color bands.

In some embodiments, an emission wavelength of each LED is selected such that a white and/or pinkish surface on healthy tissue and a red surface on non-healthy tissue can be visible identified and distinguished from each other.

In some embodiments, the at least one imaging sensor and plurality of light emitters are arranged on a plate and oriented in a same direction.

In some embodiments, the hyperspectral imaging system includes a lens system, which is a fixed lens system, detachable lens system, replaceable lens system or an interchangeable lens system. In some aspects, the lens system has at least one lens with a field of view (FOV) in a range of at least about 90 degrees and less than about 360 degrees or about 120 degree to about 180 degree. In some aspects, the hyperspectral imaging system comprises an optical lens, an optical filter, a dispersive optic system, or a combination thereof. In some aspects, the hyperspectral imaging system comprises a first optical lens, a second optical lens, and a dichroic mirror/beam splitter. In some aspects, the hyperspectral imaging system comprises an optical lens, a dispersive optic, and wherein the at least one imaging sensor is an optical detector array.

In some embodiments, the at least one imaging sensor is positioned in an off-centered position with respect to a central axis of the imaging capsule. In some aspects, the at least one imaging sensor is positioned from about 10 degrees to about 35 degrees off the central axis. In some aspects, the hyperspectral imaging system further comprises an optical filtering system placed between an optical inlet of the capsule and the at least one imaging sensor. In some aspects, the optical filtering system includes a denoising filter, such as a median filter.

In some embodiments, the imaging capsule comprises a capsule cover, wherein the capsule cover has a texture on an external surface. In some aspects, the texture comprises at least one dimple, and wherein the at least one dimple is configured such that a patient can easily swallow the tethered imaging capsule. In some aspects, the texture comprises at least one channel, and wherein the at least one channel is configured such that a patient can easily swallow the tethered imaging capsule.

In some embodiments, a display is operably coupled with the hyperspectral processing system, wherein the illumination system is calibrated for the at least one imaging sensor to display the imaged target on the display.

In some embodiments, the capsule includes a control system (e.g., in the illumination system or hyperspectral imaging system) configured to control the sequence of different lighting illuminations and imaging of the at least one imaging sensor.

In some embodiments, the hyperspectral processing system includes a control system, memory and a display, wherein the control system is configured for causing generation of the multispectral reflectance data cube, storage of the multispectral reflectance data cube in the memory, and displaying the multispectral reflectance data cube or image representation thereof on the display.

In some embodiments, the at least one optical detector has a configuration that: detects target electromagnetic radiation absorbed, transmitted, refracted, reflected, and/or emitted by at least one physical point on the target, wherein the target radiation comprises at least two target waves, each target wave having an intensity and a unique wavelength; detects the intensity and the wavelength of each target wave; and transmits the detected target electromagnetic radiation, and each target wave detected intensity and wavelength to the hyperspectral processing system. In some aspects, the hyperspectral processing system has a configuration that: forms a target image of the target using the detected target electromagnetic radiation, wherein the target image comprises at least two pixels, and wherein each pixel corresponds to one physical point on the target; forms at least one intensity spectrum for each pixel using the detected intensity and wavelength of each target wave; and generates the multispectral reflectance data cube from the at least one intensity spectrum for each pixel. In some aspects, the hyperspectral processing system has a configuration that: transforms the formed intensity spectrum of each pixel using a Fourier transform into a complex-valued function based on the intensity spectrum of each pixel, wherein each complex-valued function has at least one real component and at least one imaginary component; applies a denoising filter on both the real component and the imaginary component of each complex-valued function at least once so as to produce a denoised real value and a denoised imaginary value for each pixel; forms one phasor point on a phasor plane for each pixel by plotting the denoised real value against the denoised imaginary value of each pixel; maps back the phasor point to a corresponding pixel on the target image based on the phasor point's geometric position on the phasor plane; assigns an arbitrary color to the corresponding pixel based on the geometric position of the phasor point on the phasor plane; and generates an unmixed color image of the target based on the assigned arbitrary color. In some aspects, the hyperspectral processing system has a configuration that displays the unmixed color image of the target on a display of the hyperspectral processing system. In some aspects, the hyperspectral processing system uses only a first harmonic or only a second harmonic of the Fourier transform to generate the unmixed color image of the target. In some aspects, the hyperspectral processing system uses only a first harmonic and a second harmonic of the Fourier transform to generate the unmixed color image of the target. In some aspects, the target radiation comprises at least one of: fluorescent wavelengths; or at least four wavelengths. In some aspects, hyperspectral processing system is configured to form the unmixed color image of the target at a signal-to-noise ratio of the at least one spectrum in the range of 1.2 to 50. In some aspects, the hyperspectral processing system forms the unmixed color image of the target at a signal-to-noise ratio of the at least one spectrum in the range of 2 to 50. In some aspects, the hyperspectral processing system has a configuration that uses a reference material to assign an arbitrary color to each pixel.

In some embodiments, the hyperspectral processing system has a configuration that uses a reference material to assign an arbitrary color to each pixel, and wherein the unmixed color image of the reference material is generated prior to the generation of an unmixed color image of the target. In some aspects, the hyperspectral processing system has a configuration that uses a reference material to assign an arbitrary color to each pixel, wherein the unmixed color image of the reference material is generated prior to the generation of an unmixed color image of the target, and wherein the reference material comprises a physical structure, a chemical molecule, a biological molecule, a physical change and/or biological change caused by disease, or any combination thereof.

In some embodiments, the illumination system and hyperspectral imaging system are cooperatively configured to: illuminate a reference target with a first lighting illumination; image the reference target during the first lighting illumination; illuminate the reference target with a second lighting illumination that is different than the first lighting illumination; image the reference target during the second lighting illumination; illuminate the reference target with a third lighting illumination that is different from the first lighting illumination and second lighting illumination; and image the reference target during the third lighting illumination. In some aspects, the third lighting illumination is a white light illumination. In some aspects, the reference target includes a color standard image. In some aspects, the first lighting illumination, second lighting illumination, and third lighting illumination each includes illumination by at least two LEDs.

In some embodiments, the hyperspectral processing system has a configuration that: obtains a spectrum for each pixel of the images; and generates a transformation matrix from the spectrum of each pixel.

In some embodiments, the illumination system and hyperspectral imaging system are cooperatively configured to: illuminate the target with the first lighting illumination; image the target during the first lighting illumination; illuminate the target with the second lighting illumination; image the target during the second lighting illumination; illuminate the target with the third lighting illumination; and image the reference target during the third lighting illumination. In some aspects, the hyperspectral processing system has a configuration that generates the multispectral reflectance data cube from the transformation matrix and images of the target acquired during the first lighting illumination, second lighting illumination, and third lighting illumination. In some aspects, the multispectral reflectance data cube is obtained from a pseudo-inverse method with the images of the target.

In some embodiments, a computer method can include: causing illumination of a target with an illumination system of an imaging capsule; receiving detected target electromagnetic radiation absorbed, transmitted, refracted, reflected, and/or emitted by at least one physical point on the target from at least one imaging sensor of the imaging capsule, wherein the target radiation comprises at least two target waves, each target wave having an intensity and a unique wavelength; and transmitting the detected target electromagnetic radiation and each target wave detected intensity and wavelength from the imaging capsule to a hyperspectral processing system.

In some embodiments, the computer method can include the hyperspectral processing system performing: forming a target image of the target using the detected target electromagnetic radiation, wherein the target image comprises at least two pixels, and wherein each pixel corresponds to one physical point on the target; forming at least one intensity spectrum for each pixel using the detected intensity and wavelength of each target wave; and generating the multispectral reflectance data cube from the at least one intensity spectrum for each pixel.

In some embodiments, the computer method includes the hyperspectral processing system performing: transforming the formed intensity spectrum of each pixel using a Fourier transform into a complex-valued function based on the intensity spectrum of each pixel, wherein each complex-valued function has at least one real component and at least one imaginary component; applying a denoising filter on both the real component and the imaginary component of each complex-valued function at least once so as to produce a denoised real value and a denoised imaginary value for each pixel; forming one phasor point on a phasor plane for each pixel by plotting the denoised real value against the denoised imaginary value of each pixel; mapping back the phasor point to a corresponding pixel on the target image based on the phasor point's geometric position on the phasor plane; assigning an arbitrary color to the corresponding pixel based on the geometric position of the phasor point on the phasor plane; and generating an unmixed color image of the target based on the assigned arbitrary color. In some aspects, the hyperspectral processing system is configured for causing displaying the unmixed color image of the target on a display of the hyperspectral processing system.

In some embodiments, a computer method can include: causing illumination of a reference target with a first lighting illumination emitted from an imaging capsule; acquiring an image of the reference target with the imaging capsule during the first lighting illumination; causing illumination of the reference target with a second lighting illumination emitted from the imaging capsule; acquiring an image of the reference target with the imaging capsule during the second lighting illumination; causing illumination of the reference target with a third lighting illumination emitted from the imaging capsule; and acquiring an image of the reference target with the imaging capsule during the third lighting illumination. In some aspects, the computer method can include: obtaining a spectrum for each pixel of the images; and generating a transformation matrix from the spectrum of each pixel. In some aspects, the computer method can include: causing illumination of the target with the first lighting illumination from the imaging capsule; acquiring an image of the target with the imaging capsule during the first lighting illumination; causing illumination of the target with the second lighting illumination from the imaging capsule; acquiring an image of the target with the imaging capsule during the second lighting illumination; causing illumination of the target with the third lighting illumination from the imaging capsule; and acquiring an image of the reference target with the imaging capsule during the third lighting illumination. In some aspects, the hyperspectral processing system has a configuration that generates the multispectral reflectance data cube from the transformation matrix and images of the target acquired during the first lighting illumination, second lighting illumination, and third lighting illumination. In some aspects, the multispectral reflectance data cube is obtained from a pseudo-inverse method with the images of the target.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes a schematic representation of a capsule hyperspectral system including an imaging capsule and a hyperspectral processing system.

FIG. 1B includes a cross-sectional schematic representation of an embodiment of an imaging capsule.

FIG. 1C includes a cross-sectional schematic representation of an embodiment of an imaging capsule.

FIG. 1D includes an illustration of an imaging capsule tethered to a drone.

FIG. 1E includes an illustration of an imaging capsule configured as a ground vehicle.

FIG. 1F includes an illustration of an imaging capsule tethered to a miniature crane.

FIG. 2A includes a schematic representation of a front end plate of the capsule having the imaging sensor and an array of LEDs.

FIG. 2B includes a schematic representation of a front end plate of the capsule having two imaging sensors and an array of LEDs.

FIG. 2C includes a schematic representation of a tethered end plate of the capsule having the imaging sensor and an array of LEDs.

FIG. 2D includes a schematic representation of a tethered end plate of the capsule having two imaging sensors and an array of LEDs.

FIG. 3A includes a schematic representation of a side plate of the capsule having the imaging sensor and an array of LEDs.

FIG. 3B includes a schematic representation of a side plate of the capsule having two imaging sensors and an array of LEDs.

FIG. 4A includes a tether end view of an embodiment of a capsule having indentations in a textured cover.

FIG. 4B includes a side view of an embodiment of a capsule having indentations in a textured cover.

FIG. 4C includes a tether end view of an embodiment of a capsule having channels in a textured cover.

FIG. 4D includes a side view of an embodiment of a capsule having channels in a textured cover.

FIG. 5 includes a flow chart of a protocol for using the imaging capsule and hyperspectral processing system to convert images into a hyperspectral unmixed color image.

FIG. 6 includes a schematic representation of a workflow for generating a multispectral reflectance data cube.

FIG. 7A includes an image that shows the esophagus under normal white light illumination (e.g., representation of the multispectral reflectance data cube).

FIG. 7B includes an image that shows the esophagus in a false-color hyperspectral phasor image.

FIG. 7C includes a graph that shows the corresponding G-S histogram (e.g., phasor plot) of the esophagus.

FIG. 8A includes an image that shows intestine under normal white light illumination (e.g., representation of the multispectral reflectance data cube).

FIG. 8B includes an image that shows the intestine in a false-color hyperspectral phasor image.

FIG. 8C includes a graph that shows the corresponding G-S histogram (e.g., phasor plot) of the intestine.

FIG. 9 includes a schematic representation of a computing device that can be used in the systems and methods of the invention.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

This invention relates to a miniature, low-cost, tethered endoscope that has been developed for colored light and white light and hyperspectral-based screening of tissues, such as throat tissues. The tethered endoscope can be swallowed so that it can be used to visualize and diagnose esophageal diseases. This tethered imaging capsule, which may be designed for single use or a limited number of uses, may be intended for use by medical assistants, nurses or doctors in primary health care situations before referral to a specialist (e.g. esophageal endoscopy by a gastroenterologist). The technical advantages of this design provide improved overall efficacy of the screening process for esophageal diseases. However, the imaging capsule may not be tethered, or it may be coupled with a machine, such as a drone, ground vehicle, or crane, as well as others. The sizing of the capsule is small enough to be swallowed, and thereby the size of the machines can be equally small to fit into small spaces.

An exemplary capsule hyperspectral system may comprise a tethered imaging capsule, a tether, a light illumination system (e.g., colored and white lights), a hyperspectral imaging system, and a hyperspectral processing system. An exemplary light illuminating system may comprise an LED illumination system. In some aspects, the capsule can include at least three light sources to illuminate the target (“illumination source”) where two can be colored and the third is white, wherein the illumination source generates an electromagnetic radiation (“illumination source radiation”) that comprises at least one wave (“illumination wave”) or band for each of the three light sources. The capsule hyperspectral system may further include an imaging sensor (e.g., camera) and a display.

The present imaging capsule provides a significant improvement over a first generation device that has low resolution (400×400 pixel). The low resolution camera was found to be lacking the resolution and precise positioning capability in order to image suspected areas of esophageal diseases such as squamous cell carcinoma of the esophagus. The condition known as Barrett's esophagus may not clearly be visible at this low resolution. Thereby, the present imaging capsule provides an improvement to allow for high resolution from hyperspectral image processing. Now, the improved imaging capsule can provide the high resolution and precise positioning capability in order to image suspected areas of esophageal diseases, such as squamous cell carcinoma of the esophagus. The condition known as Barrett's esophagus can now be clearly visible with the high resolution imaging capsule.

The present high definition imaging capsule with hyperspectral processing can provide an integrated custom hardware illumination system that may utilize at least three LEDs for visualization and imaging in three sequential steps, preferably with two color illuminations and then a white light illumination. At least one of the LEDs is white. The number of the LEDs in the capsule can range from three LEDs to six LEDs or more, which may enable software-based hyperspectral decomposition of high resolution images (e.g., up to 1280×1080 pixels at 60 Hz frame rates) using a non-specialized CMOS-based imaging sensor. A 60 Hz frame rate or greater is used in order to minimize motion artifacts during screen capture. The imaging can have effective filtering of wavelength bandwidths of approximately 10 nm, over the visible wavelength range from about 410 nm to about 750 nm, or effective spectral range for device. For example, six LEDs may enable the identification of 32 spectral bands of the visible light spectrum.

The imaging sensor can be configured as a high definition camera with a resolution comparable to existing top-of-the-line, FDA approved, endoscopes for gastrointestinal (GI) exams (e.g. Olympus endoscopes with 1080p resolution). The high frame rate (60+ fps) may significantly reduce motion artifacts in screen captured images for detailed analysis by automated machine vision software programs or medical specialists.

A tether (e.g., wire, guidewire, cord, data cord, etc.) can be coupled to the capsule. The tether may enable a moderately experienced user to manually and precisely rotate the camera position within the esophagus for improved imaging of suspected disease areas. This tether, not only may supply power and a data transfer link to the capsule, but may also be marked on its surface with visible markings at regular intervals so that the user may accurately measure the position of suspected diseases areas in the esophagus for later, follow-up examination.

The surface of the casing of the capsule may have a texture (e.g. surface dimples, and grooves or channels not parallel to the longitudinal axis of the capsule) that may remove fluids from the front part of the device, may make it easy to swallow and to recover after the screening exam.

The capsule hyperspectral system may, for example, be intended for use for the screening of tissues in a gastro-intestinal tract after swallowing the capsule attached to the tether. The tissues can be any gastro-intestinal tract tissue, such as esophageal tissues, which may be useful for identifying esophageal diseases. The tethered capsule can be used in primary care facilities with limited access to secondary or tertiary GI specialists.

The capsule hyperspectral system can be used to visualize esophageal diseases and conditions, which may be diagnosed with this system. Some examples without limitation include Barrett's esophagus, esophageal cancer (EC), stomach/gastric cancer, gastroesophageal reflux disease (GERD), peptic ulcer disease, swallowing disorders, and esophageal varices. Since esophageal varices are a condition commonly associated with liver disease, this capsule hyperspectral system may be used in diagnosing liver diseases.

In some embodiments, the capsule hyperspectral system can be configured as a low-cost, easy to use, HD-TIC system. The HD-TIC system may be intended for annual or regular periodic health care screening of the esophagus for dysplasia due to esophageal cancers (adenocarcinoma and squamous cell carcinoma) and associated symptoms of liver diseases (e.g. esophageal varices, other signs of portal hypertension).

However, the capsule hyperspectral system may be used in other environment, such as in crevasses, wells, small tunnels or conduits, air flow pathways, ventilation systems, in nature, or in any other place or use. The target can be any target object for illuminating and imaging.

The description of the capsule hyperspectral system provided herein can be applied to any environment for illumination and imaging as described.

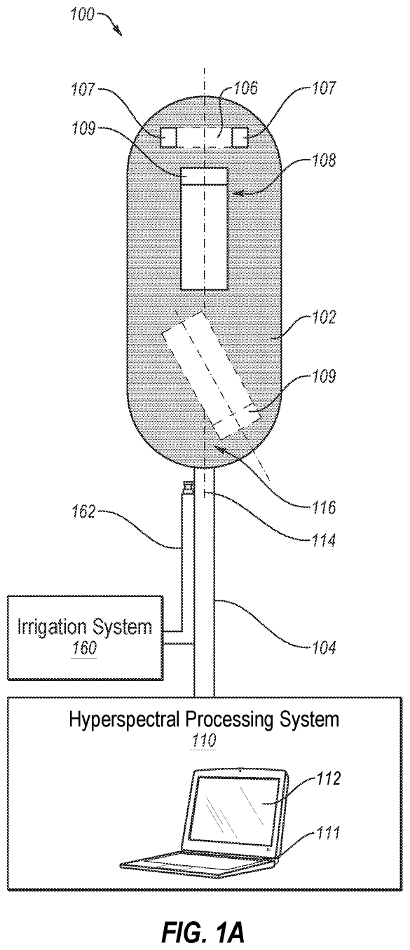

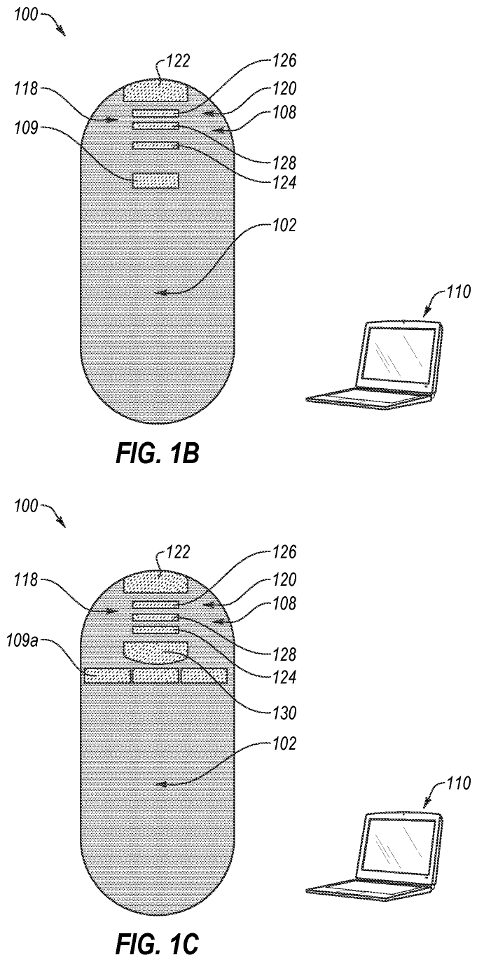

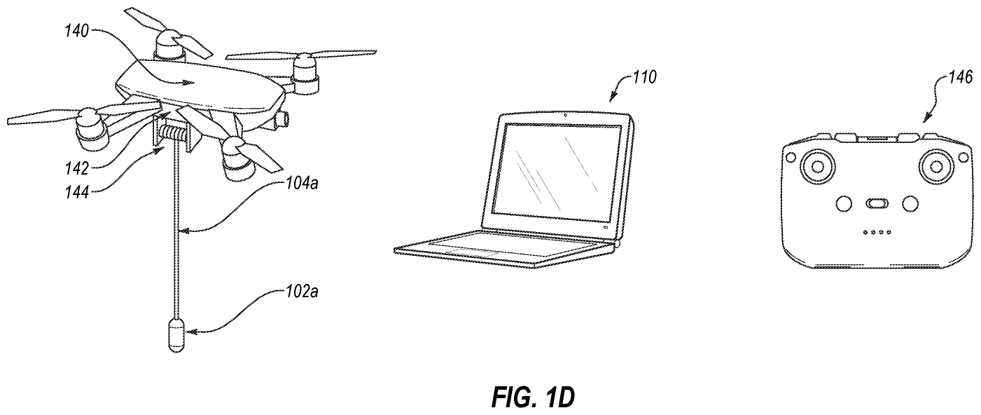

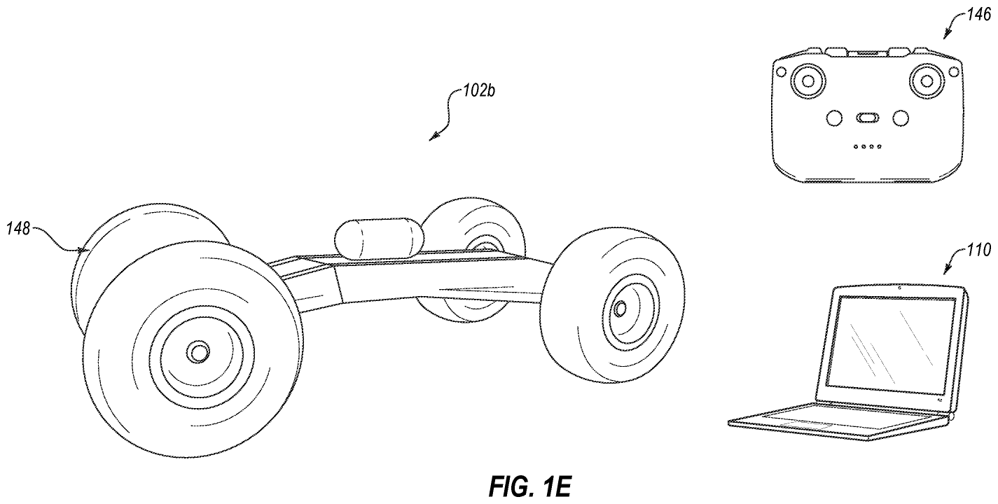

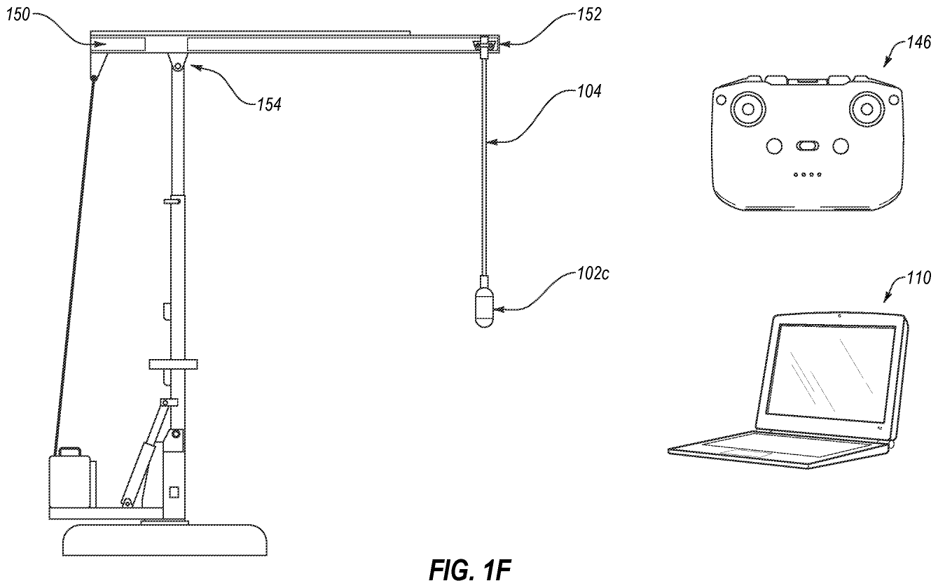

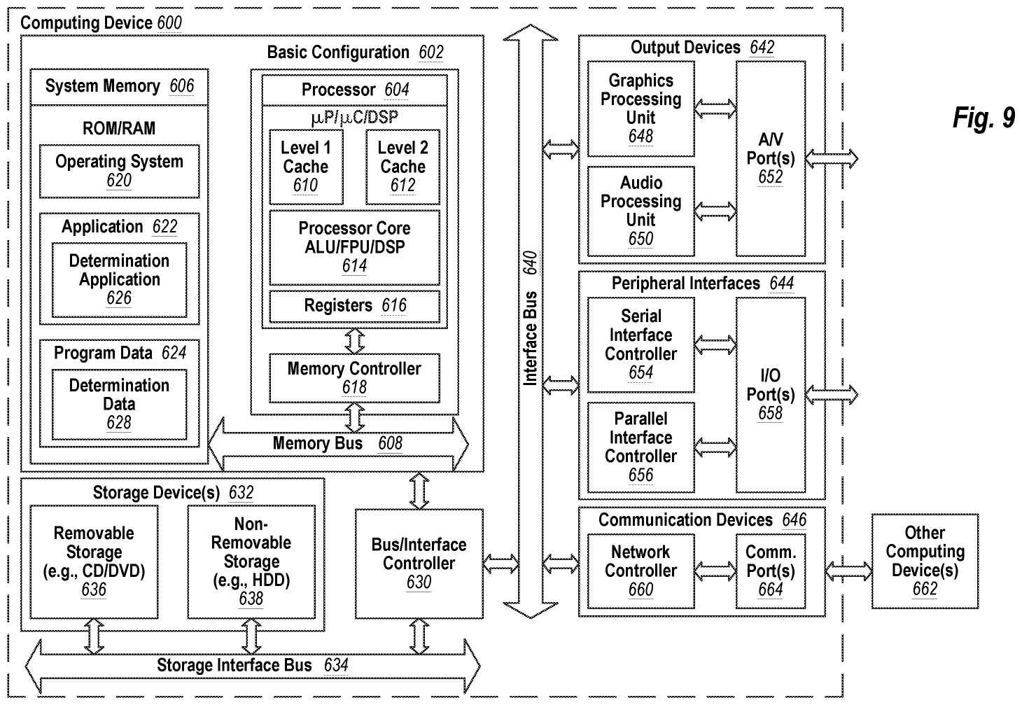

FIG. 1A illustrates an embodiment of a capsule hyperspectral system 100. The capsule hyperspectral system 100 is shown to include a tethered imaging capsule 102 attached at an end to a tether 104. The capsule 102 includes a light illumination system 106 with at least three light emitters 107 configured for emitting various colors (e.g., red, blue, green, yellow, orange, purple, etc.) as well as white light. The capsule hyperspectral system 100 can include the imaging capsule 102 having a hyperspectral imaging system 108 with at least one imaging sensor 109. The tether 104 is operably coupled with a hyperspectral processing system 110 that has at least one processor 111, which can be at least part of one or more computers, such as shown in FIG. 9.

While the capsule 102 can be tethered to the hyperspectral processing system 110, it can also be decoupled or not tethered. In this instances, the capsule 102 can include a memory card that can plug into the hyperspectral processing system 110, or the capsule 102 can directly plug into the hyperspectral processing system 110.

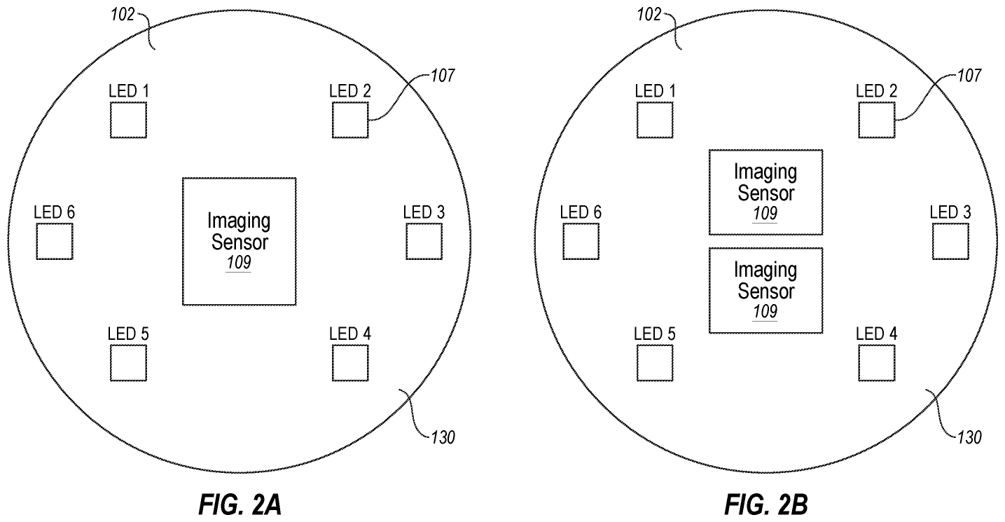

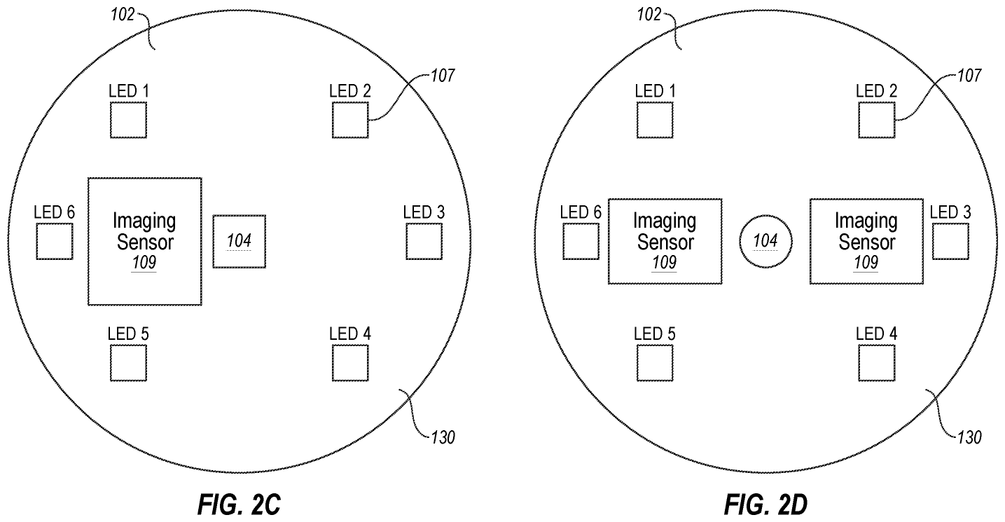

The illumination system 106 can include an LED illumination system that includes three or more LEDs as the light emitters 107. The LEDs may be calibrated for the camera (e.g., imaging sensor 109) of the imaging capsule 102. Also, the LEDs may be tailored for the display 112, such that an image of the illuminated tissue (e.g., esophagus) may freely be displayed on any display system having a display 112. In some aspects, the imaging sensor 109 is centered on an axis of the tethered imaging capsule 102, as shown (e.g., FIG. 2A). In some aspects, the imaging sensor 109 may have an off-centered position with respect to the axis 114 of the tethered imaging capsule (e.g., FIG. 2B). For example, the imaging sensor 109 may be positioned in an off-centered at an angle 116 with respect to the axis of the tethered imaging capsule, wherein the camera is positioned 35 degrees off the axis, +/−1%, 2%, 5%, 10%, or 20%. That is, the light directed from the imaging sensor 109 can be at the angle 116 from the axis 114.

In some embodiments, the capsule hyperspectral system 100 may further include a uniformly arranged array of a plurality of LEDs. For example, a design can include at least or up to six LEDs (e.g., three pairs) for uniform illumination, such as being located around the imaging sensor(s). The emission wavelength of the LEDs may be selected such that white/pinkish surface on healthy esophagus and red surface on non-healthy esophagus can easily be identified. However, it should be recognized that at least three LEDs can perform the actions described herein with three different lighting conditions. The three different lighting conditions can use two lights for each condition, thereby using six lights. The pair of lights for each lighting condition can improve with light coverage for improved imaging. While three pairs of LEDs are a good example, there can be six different light colors. Alternatively, there can be at least two different pairs of colored lights and a pair of white lights, which may include a pair of the same color (both green) or a pair of different colors (e.g., red and blue). The number of LEDs and the different colors is not limited herein.

In some embodiments, the capsule 102 can provide a color image to any type of display system. The light emitters can illuminate with any light color combination during imaging, which can change, and which can be displayed.

FIG. 1A also shows an irrigation system 160 that can include a pump that supplies irrigation fluid (e.g., water) to the site being imaged to clean the site. The cleaning can remove debris or body materials to improve imaging. The irrigation system 160 can include an irrigation conduit 162 with an opening at or near the capsule 102 to emit fluid around the capsule 102. The conduit 162 can be around the tether 104 or otherwise associated with it.

FIGS. 1B-1C illustrate the hyperspectral imaging system 108, which may include an optics system 118. The optics system 118 may include at least one optical component. The at least one optical component may comprise at least one optical detector, such as an imaging sensor 109 and optionally a lens system 120 (e.g., one or more) optically coupled with the imaging sensor 109. The optical detector can be any optical detector, which can be a photodiode or other imaging sensor. A camera imaging device (e.g., photomultiplier tube, photomultiplier tube array, digital camera, hyperspectral camera, electron multiplying charge coupled device, sci-CMOS, or combination thereof, etc.) may be used for the detector. The optical detector may have a configuration that: detects target electromagnetic radiation absorbed, transmitted, refracted, reflected, and/or emitted (“target radiation”) by at least one physical point on the target, the target radiation comprises at least two target waves (“target waves”), each wave having an intensity and a different wavelength; detects the intensity and the wavelength of each target wave; and transmits the detected target radiation, and each target wave's detected intensity and wavelength to the hyperspectral processing system 110. The hyperspectral processing system 110 is described in more detail below.

The at least one optical component can include an optical lens 122, an optical filter 124, a dispersive optic 130, or a combination thereof. The at least one optical component further may comprise a first optical lens 126, a second optical lens 128, and an optical filter 124, which can be configured as a dichroic mirror/beam splitter. The at least one optical component may further comprise an optical lens 122, a dispersive optic 130, and wherein at least one imaging sensor 109 is an optical detector array 109a.

In some embodiments, the at least one optical component may include an optical filtering system having at least one optical filter 124 placed between the target to be imaged and the at least one imaging sensor 109. The target radiation emitted from the target may include an electromagnetic radiation emitted by the target. In some aspects, the electromagnetic radiation emitted by the target comprises fluorescence. In some aspects, a denoising filter of an optical filter 124 may comprise a median filter.

As shown in FIG. 1C, the capsule hyperspectral system 100 may further comprise a lens system. The lens system of one or more lenses can include a lens 122 with a field of view (FOV) in a range of about 120 degree to a greater than 180 degree, or in a range of about 120 degree to about 190 degree, or in a range of about 120 degree to about 180 degree. The lens system may be configured to image the backside of the gastroesophageal sphincter. In some aspects, the lens system can be configured as a replaceable lens system or an interchangeable lens system. Also, the lens system may be placed at distal end of the capsule hyperspectral system, such as at the capsule. The lens system may include more than one lens.

The capsule 102 can include an illumination system and detection system such as described in WO 2018/089383, which is incorporated herein by specific reference in its entirety, such as in FIGS. 14-21.

FIG. 1D illustrates an imaging capsule 102a configured to be used from a drone 140 (e.g., unmanned arial vehicle). The drone 140 includes a tethering system 142 having a mechanical part 144 (e.g., pully, spindle, winch, etc.) to raise or lower the capsule 102a on a tether 104a. The tether 104a can be lengthened or shortened as needed or desired for use in imaging. For example, the drone 140 may not be able to lower, but then the mechanical part 144 can lower the tether 104a to lower the capsule 102a. The capsule 102a and/or drone 140 can include a controller (e.g., computer) as described herein that can operate the drone 140 and the capsule 102a for imaging purposes. The drone 140 and/or capsule 102a may include a transceiver that can transmit data to a hyperspectral processing system 110, such as wireless data transmission. The tether 104a may provide data lines for data communication between the drone 140 and capsule 102a, or each can include a transceiver for wireless data communications. Also, a remote controller 146 may also be used to control the drone 140, where the remote controller 146 can wirelessly control operation of the drone 140. The remote controller 146 can communicate directly with the drone 140 or via a control module, which can be part of the computer of the hyperspectral processing system 110.

FIG. 1E illustrates an imaging capsule 102b configured to be used from a ground vehicle 148 (e.g., unmanned ground vehicle, remote control). The capsule 102b is mounted to the vehicle 148 in any way, and may serve as a body of the vehicle. The vehicle 148 may be the size of a normal RC car, or miniature to take advantage of the small size of the capsule 102b, which can be a swallowable size. The small ground vehicle 148 can be used to access small places that are not able to be accessed by a human or larger equipment. The capsule 102b and/or vehicle 148 can include a controller (e.g., computer) as described herein that can operate the vehicle 148 and the capsule 102b for imaging purposes. The vehicle 148 and/or capsule 102b may include a transceiver that can transmit data to a hyperspectral processing system 110, such as wireless data transmission. Also, a remote controller 146 may also be used to control the vehicle 148, where the remote controller 146 can wirelessly control operation of the vehicle 148. The remote controller 146 can communicate directly with the vehicle 148 or via a control module, which can be part of the computer of the hyperspectral processing system 110. Alternatively, the ground vehicle can be configured as a tank, dog, insect, or spider, where wheels, treads, legs, and other moving members can propel the vehicle.

FIG. 1F illustrates an imaging capsule 102c configured to be used from a small crane 150 (e.g., winch). The crane 150 includes a tethering system 152 having a mechanical part 154 (e.g., winch, etc.) to raise or lower the capsule 102c on a tether 104b. The tether 104b can be lengthened or shortened as needed or desired for use in imaging. For example, the crane 150 may not be able to lower when placed in a location for use (e.g., mounted to a well), but then the mechanical part 154 can lower the tether 104b to lower the capsule 102c. The capsule 102c and/or crane 150 can include a controller (e.g., computer) as described herein that can operate the crane 150 and the capsule 102c for imaging purposes. The crane 150 and/or capsule 102c may include a transceiver that can transmit data to a hyperspectral processing system 110, such as wireless data transmission. The tether 104b may provide data lines for data communication between the crane 150 and capsule 102c, or each can include a transceiver for wireless data communications. Also, a remote controller 146 may also be used to control the crane 150, where the remote controller 146 can wirelessly control operation of the crane 150. The remote controller 146 can communicate directly with the crane 150 or via a control module, which can be part of the computer of the hyperspectral processing system 110.

FIG. 2A shows a front view of the imaging capsule 102 having the imaging sensor 109 and six LED illuminators (e.g., light emitters 107) arranged there around. In this example, the front view looks down the axis 114 of the capsule 102. The light emitters 107 are arranged on a patterned plate 130, where the light emitters 107 can be any of the colors and combinations as described herein. The light emitters 107 can include white light LEDs and a specific combination of narrow band color LEDs. During manufacturing, the arrangement of the imaging sensor 109 and light emitters 107 on the plate 130 can be changed so that specific variations of the high definition imaging capsule are possible (e.g., a for white light imaging and/or a for hyperspectral imaging). The pin-out alignment of this plate 130 matches the wiring to the tether and/or the capsule. The light emitters 107 can either be illuminated all at once, in pairs, or sequentially (e.g., in sequence of pairs), as selected by the user in software settings and electronic-controlled switching. For white light illumination, the white LEDs are all illuminated at once. For hyperspectral imaging, the colored LEDs and optionally white LEDs are illuminated in a sequence that is synchronized with the frame rate of the imaging sensor 109. In an example, this arrangement allows the generation of a hyperspectral data cube that can, after post-processing by the hyperspectral decomposition method, be used to identify regions of dysplasia in the esophagus via color differences (e.g. shifts or differences in the response wavelength of illuminated regions of the esophagus indicating possible pre-cancerous or cancerous lesions). However, it should be recognized that any imaging use of a tissue or any other object or environment can be performed. The schematic of the imager and LEDs in FIG. 2A shows a single imaging sensor 109. The schematic of the imager and LEDs in FIG. 2B shows a pair of imaging sensors 109 off center from a central axis.

The light emitters 107 may each comprise a coherent electromagnetic radiation source. The coherent electromagnetic radiation source may comprise a laser, a diode, a two-photon excitation source, a three-photon excitation source, or a combination thereof. The light emitter radiation may comprise an illumination wave with a wavelength in the range of 300 nm to 1,300 nm. The illumination source radiation may comprise an illumination wave with a wavelength in the range of 300 nm to 700 nm. The illumination source radiation may comprise an illumination wave with a wavelength in the range of 690 nm to 1,300 nm.

In an example, LED 1 can be blue, LED 2 can be orange, LED 3 can be green, LED 4 can be red, and LED 5 and LED 6 are both white. During imaging, two colored lights are illuminated, which can be any combination of these two lights. Then in the next imaging two different lights are illuminated. Then in the third imaging, the two white LEDs are used. This helps to analyze each part of the light spectrum with the different lights. This helps construct the colors of the target. In another example, only three LEDs are used. There can be two colored LEDs and one white LED.

FIG. 2B shows a front view of an embodiment of the tethered imaging capsule 102 with multiple imaging sensors 109 surrounded by the light emitters 107 in an array on the plate 130. The imaging sensors 109 are off center from a central axis. The imaging sensors 109 may be oriented parallel or the surfaces thereof can be at an angle so that they both point to a common point on the central axis.

FIG. 2C shows an end view of the tethered imaging capsule 102 having the imaging sensor 109 surrounded by the light emitters 107, which are LED illuminators. The light emitters 107 are arranged on the patterned plate 130, and can include the combinations of white light LEDs or narrow band color LEDs. During manufacturing, the configuration or pattern/arrangement of the imaging sensor(s) 109 and light emitters 107 on the plate 130 can be changed so that specific variations of the high definition imaging capsule are possible (e.g., for white light imaging or a for hyperspectral imaging). The pin-out alignment of this LED plate 130 matches the wiring to the tether 104 from the capsule 102. The light emitters 107 can either be illuminated all at once, in pairs or sequentially, as selected by the user in software settings and electronic-controlled switching. For white light illumination, the white LEDs are all illuminated at once. For hyperspectral imaging, the LEDs are illuminated in a sequence that is synchronized with the frame rate of the imaging sensor 109. This allows the generation of a hyperspectral data cube that can, after post-processing by the hyperspectral decomposition method, to characterize objects, such as identify regions of dysplasia in the esophagus, via color differences (e.g. shifts or differences in the response wavelength of illuminated regions of the esophagus indicating possible pre-cancerous or cancerous lesions). A schematic of the imager and LEDs in FIG. 2C shows a single imaging sensor 109 offset from the tether 104.

FIG. 2D shows a plate 130 having multiple imaging sensors 107 offset from the tether 104 and surrounded by the imaging sensors 109.

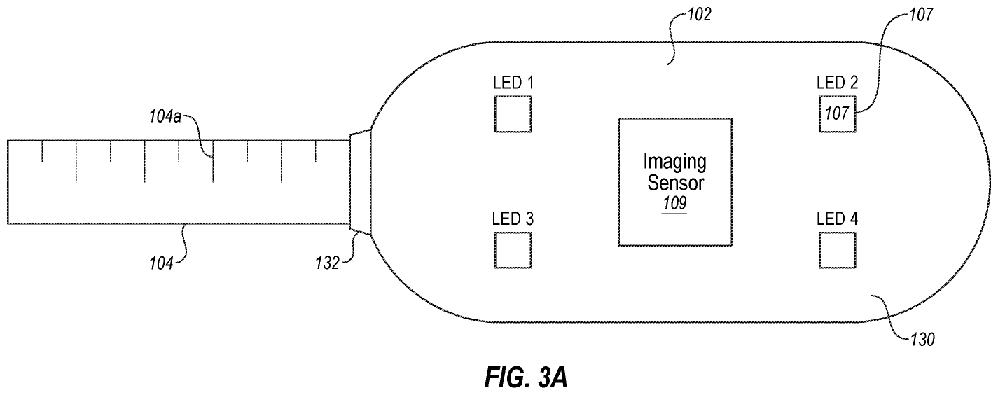

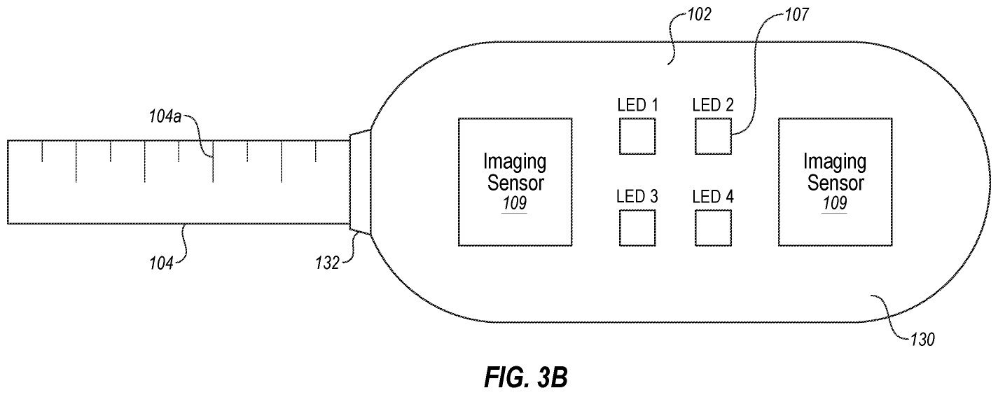

FIG. 3A shows a side view of a tethered imaging capsule 102 having the imaging sensor 109 surrounded by the light emitters 107, which are LED illuminators. The light emitters 107 are arranged on a patterned plate 130 that can be in any of the colors or color combinations described herein, such as having white light LEDs and a specific combination of narrow band color LEDs. During manufacturing, this plate 130 can be changed so that specific variations of the high definition imaging capsule are possible. (e.g. white light imaging or a hyperspectral imaging). The pin-out alignment of this LED plate 130 matches the wiring to the tether from the capsule. The light emitters 107 can either be illuminated all at once, in pairs, or sequentially, as selected by the user in software settings and electronic-controlled switching. For white light illumination, the white LEDs are all illuminated at once. For hyperspectral imaging, the LEDs are illuminated in a sequence that is synchronized with the frame rate of the imaging camera. A schematic of the imager and LEDs in FIG. 3A shows a single imaging sensor 109 and FIG. 3B shows multiple imaging sensors 109.

In some embodiments, the tether 104 can be any type of suitable tether to attach the capsule device 102 to the rest of the system. The tether 104 can have any cross-sectional shape in some embodiments. FIG. 2C shows a square cross-sectional profile and FIG. 2D shows a circular cross-sectional profile; however, other cross-sectional shapes may be used. The tether 104 can have a cross-sectional shape that helps in providing a reference to the location of the capsule in the body. For example, each surface of a shape can have an identifier that can be observed and tracked to know the orientation of the capsule 102 and cameras thereof. Rotating to the next body surface of the tether 104 being up can provide an angle of rotation based on the number of sides, such as 90 degrees for a square. In some aspects, the tether may be a non-circular tether 104, which may be a polygon, such as a triangle, rectangle, square, pentagon or other polygon, where the square cross-sectional profile is illustrated. The non-circular tether 104 can be configured to create an angular reference for a user during use to know where the capsule is, such as each surface being labeled for tracking and observing the up facing surface. Then, that angular reference can be used for so that the user can precisely rotate the camera for a follow-up study to place it in the same location. The tether 104 may include markings 104a (e.g., ruler lines, inches, centimeters, etc.) on its surface configured to determine position of the tethered imaging capsule 102 when the tethered imaging capsule is deployed. The non-circular tether 104 that allows for precise manual rotation and positioning of the capsule with respect to the side walls of the esophagus.

In some embodiments, the tether 104 is tethered to the capsule 102. The coupling of the tether 104 to capsule 102 can include a mechanical portion to make the mechanical coupling and can include an optical and/or electronic coupling for data transmission from the capsule to the system or vice versa. The capsule hyperspectral system 100 may include a mechanical coupling that forms a semi-rigid connection 132 between the tether 104 and the tethered imaging capsule 102 to be able to withstand manual manipulation to position the capsule. The semi-rigid connection can be via epoxy or other material if coupling. Also, silicone connections can be used to provide the semi-rigid connection. The semi-rigidity provides for flexibility so that the tether does not break from the capsule 102.

In some embodiments, the imaging capsule may include a capsule cover (e.g., silicone). In some aspects, the capsule cover has a texture on its surface. In some examples, the texture comprises a dimple and/or a channel or other feature. The texture may be configured such that a patient can easily swallow the tethered imaging capsule. That is, the texture can help the patient swallow the capsule. The cover can be applied to capsules adapted for swallowing by a patient; however, the other types of capsules for environmental or object imaging may also have a cover.

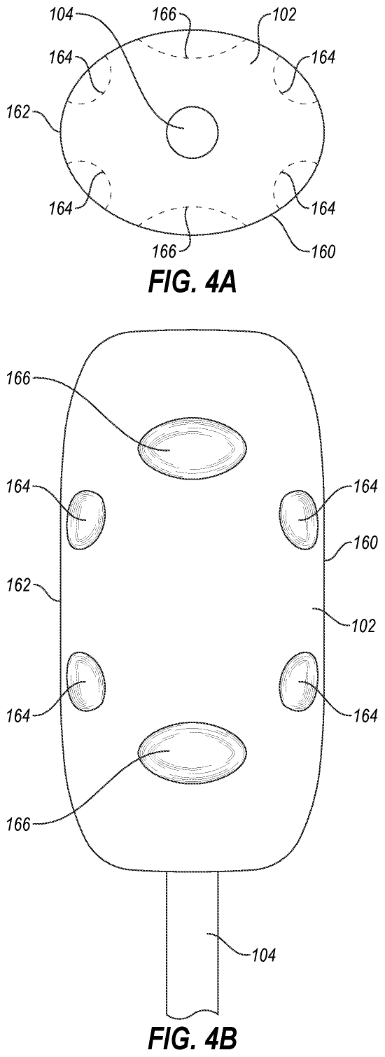

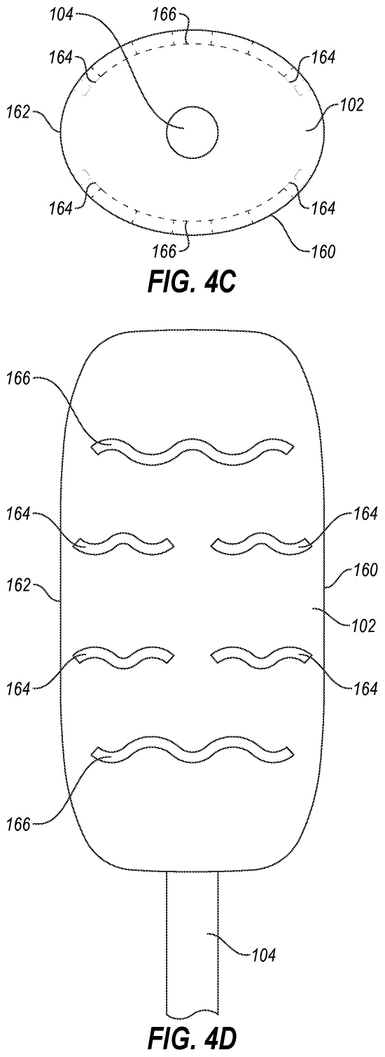

FIG. 4A shows a bottom view of the tethered end of the capsule 102 and FIG. 4B shows a side view of the tethered capsule 102, where the capsule 102 has a cover 160 with a textured surface 162. The textured surface 162 of the capsule cover 160 can be used to facilitate ease of swallowing and to direct liquid drainage away from the imager end of the capsule 102. By strategic placement of the textured surface structures, this effectively creates hydrophilic regions on the capsule cover 160 which promotes liquid droplet accumulation. This acts to effectively suction water away from the direction of the lens or lenses of the imager part of the capsule 102. This figure shows an exemplary texture: an array of small dimples 164 and large dimples 166.

FIG. 4C shows a bottom view of the tethered end of the capsule 102 and FIG. 4D shows a side view of the tethered capsule 102, where the capsule 102 has a cover 160 with a textured surface 162. The textured surface 162 of the capsule cover 160 is provided to facilitate ease of swallowing and to direct liquid drainage away from the imager end of the capsule 102. By strategic placement of the textured surface 162 structures, this effectively creates hydrophilic regions on the capsule cover 160 which promotes liquid droplet accumulation. This acts to effectively suction water away from the direction of the lens or lenses of the imager. This figure shows an exemplary texture: an array of long channels 166 and short raised channels 164.

The capsule systems may have integrated hardware and software components. The hardware may, for example, include a miniature high definition camera (e.g., imaging sensor) with custom illumination (e.g., light emitters, such as LED). This illumination may allow for the use of a hyperspectral post-processing technique in order to assist a non-GI-medical specialist in the early detection of signs of esophageal disease.

The capsule systems may have the following designed functions and advantages. The capsule imaging system can provide high definition video with image resolutions comparable to the latest generation of Olympus and Pentax endoscopes that cost 100× more. The tether may be a strong, flexible, whether circular or non-circular tether (e.g., polygon, such as flat rectangle). The configuration may make the tether (i) easy for the patient to swallow the capsule, (ii) for easy retrieval of the capsule after use (e.g. examination by only one nurse, without an assistant) (iii) for the medical professional to manually, in a controlled mechanical and analog manner, to rotate and position the capsule at precise locations in the upper GI tract (e.g. the esophagus and upper stomach). The capsule imaging system may have options for different lenses (e.g. 120 degree FOV to a +170 degree FOV or about 140 degrees FOV with different magnifications) to optimally screen throat tissues, such as for different types of cancers as well as for imaging objects or environments that are difficult to access. Different lens systems can be interchangeable so that different needs can use different lens systems. The capsule illumination systems may be configured with the proper light emitters to emit broadband white light for normal illumination or with a custom LED configurations for illumination suitable for hyperspectral analysis. This may make the video images compatible with existing hyperspectral analysis software (e.g., see incorporated references).

In some embodiments, the swallowable version of the capsule does not require or use an additional corn starch-based palatant to make the capsule palatable. The swallowable capsule can include a textured cover on the outer capsule casing in order to make it easier to swallow. The capsule can have an increased diameter while adding dimples or channels and optionally additional texture to the cover in order to make the capsule easier to swallow without a palatant.

The hyperspectral processing system can be operably coupled to the imaging capsule via the tether or wireless data communications. As such, the tether can include data transmission lines, such as optical and/or electrical. However, in one aspect, the hyperspectral imaging system can include a battery to operate the components in the capsule and include a transceiver for transmitting and receiving data with the hyperspectral processing system. Also, the capsule can have memory to save the images or video that are acquired during use, which can then be downloaded into the hyperspectral processing system.

The hyperspectral processing system can be or include a computer with specialized software that is capable of performing the imaging as described herein. As such, FIG. 9 illustrates an example of the hardware components of the hyperspectral processing system. The memory devices of the hyperspectral processing system can include computer-executable code that causes performance of the methods described herein in order to image a tissue with the capsule hyperspectral system.

The hardware of the capsule systems may be optimized in order to generate high quality images that may be analyzed by a medical assistant or medical specialist. The imaging hardware may also be optimized to be compatible with hyperspectral imaging and associated automated machine learning algorithms. As such, this hardware may be used with the hyperspectral systems and methods disclosed in a PCT application entitled “A Hyperspectral Imaging System,” (WO2018/089383). The contents of this application is incorporated herein in its entirety. Briefly, the hyperspectral decomposition systems and methods, which may be used together with the hyperspectral endoscopy system of this disclosure is outlined herein.

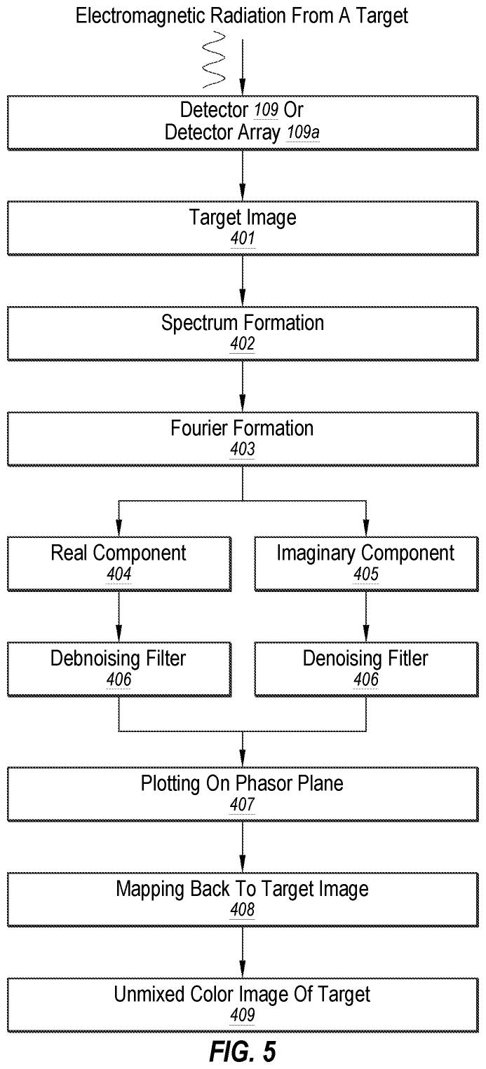

Another example of the hyperspectral imaging system is schematically shown in FIG. 5. In this example, the hyperspectral imaging system further comprises at least one detector 109 or a detector array 109a. This imaging system may form an image of a target 401 (form target image) by using the detector or the detector array. The image may comprise at least two waves and at least two pixels. The system may form an image of the target using intensities of each wave (“intensity spectrum”) 402 (spectrum formation). The system may transform the intensity spectrum of each pixel by using a Fourier transform 403, thereby forming a complex-valued function based on the detected intensity spectrum of each pixel. Each complex-valued function may have at least one real component 404 and at least one imaginary component 405. The system may apply a denoising filter 406 on both the real component and the imaginary component of each complex-valued function at least once. The system may thereby obtain a denoised real value and a denoised imaginary value for each pixel. The system may plot the denoised real value against the denoised imaginary value for each pixel, and the system may thereby form a point on a phasor plane 407 (plotting on phasor plane). The system may form at least one additional point on the phasor plane by using at least one more pixel of the image. The system may select at least one point on the phasor plane, based on its geometric position on the phasor plane. The system may map back 408 the selected point on the phasor plane to corresponding pixel on the image of the target and may assign a color to the corresponding pixel, and wherein the color is assigned based on the geometric position of the point on the phasor plane. As a result, the system may thereby generate an unmixed color image of the target 409.

The image forming system may have a configuration that: causes the optical detector to detect the target radiation and to transmit the detected intensity and wavelength of each target wave to the image forming system; acquires the detected target radiation comprising the at least two target waves; forms an image of the target using the detected target radiation (“target image”), wherein the target image comprises at least two pixels, and wherein each pixel corresponds to one physical point on the target; forms at least one spectrum for each pixel using the detected intensity and wavelength of each target wave (“intensity spectrum”); transforms the formed intensity spectrum of each pixel using a Fourier transform into a complex-valued function based on the intensity spectrum of each pixel, wherein each complex-valued function has at least one real component and at least one imaginary component; applies a denoising filter on both the real component and the imaginary component of each complex-valued function at least once so as to produce a denoised real value and a denoised imaginary value for each pixel; forms one point on a phasor plane (“phasor point”) for each pixel by plotting the denoised real value against the denoised imaginary value of each pixel; maps back the phasor point to a corresponding pixel on the target image based on the phasor point's geometric position on the phasor plane; assigns an arbitrary color to the corresponding pixel based on the geometric position of the phasor point on the phasor plane; and generates an unmixed color image of the target based on the assigned arbitrary color. The image forming system may also have a configuration that displays the unmixed color image of the target on the image forming system's display.

The image forming system may have a configuration that uses at least one harmonic of the Fourier transform to generate the unmixed color image of the target. The image forming system may be configured to use at least a first harmonic of the Fourier transform to generate the unmixed color image of the target. The image forming system may be configured to use at least a second harmonic of the Fourier transform to generate the unmixed color image of the target. The image forming system may be configured to use at least a first harmonic and a second harmonic of the Fourier transform to generate the unmixed color image of the target.

The methods of operation can include the methods recited herein. The imaging capsule can be linked to the hyperspectral processing system via the tether or wirelessly. In some embodiments, the imaging capsule can then be swallowed by a patient for imaging of their throat. The hyperspectral processing system can cause the illumination system to activate the plurality of light emitters to illuminate the esophagus. The hyperspectral processing system can cause the hyperspectral imaging system to cause the at least one sensor to image the esophagus and transmit the image data to the hyperspectral processing system. The hyperspectral processing system can then process the image data as described herein and in the incorporated references for generating images of the tissue. The image data can be used for generating a multi-spectral reflectance data cube from a series of images.

In some embodiments, the imaging capsule can then be lowered into a crevasse or well or other small opening environment for imaging thereof in regions not accessible by humans. The hyperspectral processing system can cause the illumination system to activate the plurality of light emitters to illuminate the environment or objects thereof. The hyperspectral processing system can cause the hyperspectral imaging system to cause the at least one sensor to image the environment and transmit the image data to the hyperspectral processing system. The hyperspectral processing system can then process the image data as described herein and in the incorporated references for generating images of the environment and objects therein. The image data can be used for generating a multi-spectral reflectance data cube from a series of images.

The unmixed color image of the target may be formed at a signal-to-noise ratio of the at least one spectrum in the range of 1.2 to 50. The unmixed color image of the target may be formed at a signal-to-noise ratio of the at least one spectrum in the range of 2 to 50.

The target may be any target and the environment may be any environment, whether in a living subject or within an inanimate environment. The target may be any target that has a specific spectrum of color. For example, the target may be a tissue, a fluorescent genetic label, an inorganic target, or a combination thereof. In the environment, the target can be a plant or a leaf to check for the health of the plant or readiness of crops for cultivation.

The hyperspectral imaging system may be calibrated by using a reference material to assign arbitrary colors to each pixel. The reference material may be any known reference material. For example, the reference may be any reference material wherein unmixed color image of the reference material is determined prior to the generation of unmixed color image of the target. For example, the reference material may be a physical structure, a chemical molecule, a biological molecule, a biological activity (e.g. physiological change) as a result of physical structural change and/or disease.

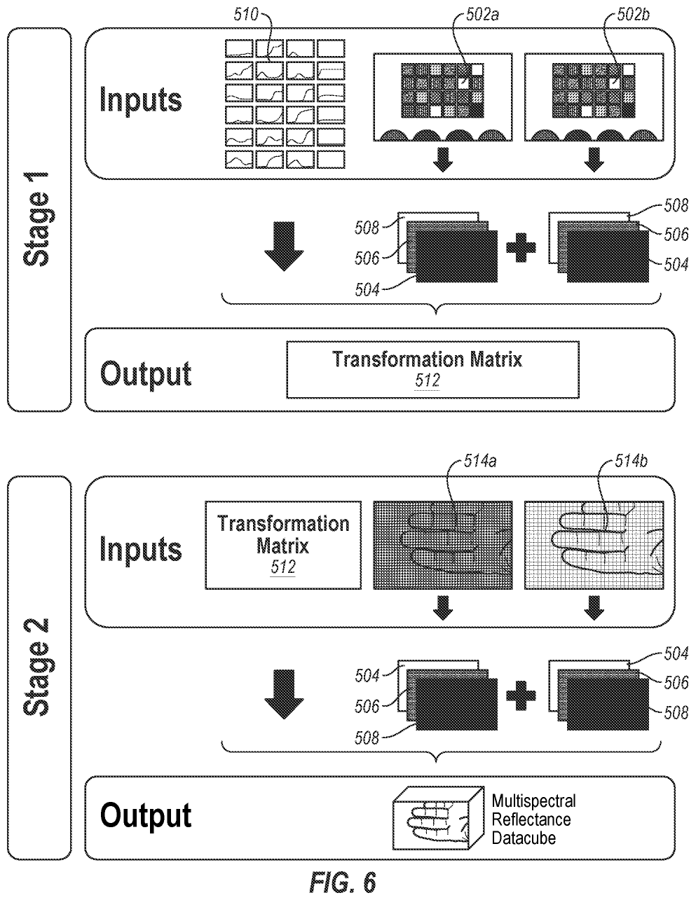

FIG. 6 shows two stages of an imaging protocol. Stage 1 includes imaging at least two color standards 502a, 502b that can be the same or different (e.g., different colors in the standard). Each color standard 502a, 502b is illuminated with a series of colors, shown as blue 504, green 506, and red 508; however, other colors can be used, such as for example the red 508 being replaced with white light illumination. Both of the color standards 502a, 502b are imaged with the same colors in the same color sequence. While each color illumination can be a single color or a single LED, better illumination can be obtained by a pair of LEDs, which can both be the same color or a color pair (e.g., red and blue). While illuminating, the three consecutive images are acquired to match the three consecutive illuminations, such as illuminate two LEDs, then take an image, then illuminate two LEDs, then take an image, then illuminate two LEDs, and then take an image.

The use of at least a pair of LEDs per illumination can help because of the spectral properties of the LEDs. The use of at least pairs of LEDs per image acquisition allows to extend the range of sampling. For example, a blue LED only would only sample the blue area and not much of the yellow are or other colored areas. If the illumination shines the blue LED with a yellow LED, then the information comes from a blue LED and a yellow LED, which is better. The second illumination and imaging step uses a red LED and green LED for imaging. The third step uses a pair of white LEDs. Now, the data has that color spectrum of the target for the different illuminations. The system knows for each one of the position targets (e.g., a pixel) what the spectrum is. For every pixel in the image, the data includes three sets of encoding information from the three sets of illumination and imaging. With the three different illuminating imaging sets of encoding data, the system then can determine that that a particular pixel corresponds to one point in the color of the target, and that one point in the color of the target has a specific spectrum as shown by the pixel spectra graphs 510, such as each graph for a unique color target on the color standards. Then, there is a matrix of three by whatever number of sets of images are taken.

The data is provided to obtain the transformation matrix 512. The protocol can find the transformation matrix, and then maximize it by multiplying it by whatever data is collected. This is the closest thing to the spectrum of 1 or the color of the target. In the color of the targets (color standards) there are a lot of different colors, which provides a lot of different spectrum 510. The process repeats the same operation for all of the different colors until it finds the matrix that works well enough for most of the spectrums 510 that are obtained. Basically, the matrix is a correction matrix from the three different illuminations from the LEDs. Once the protocol finds that matrix, that matrix is fixed as long as there is no need to change the instrument. This effectively calibrates the system with the instruments used to have a transformation matrix. The transformation matrix allows for reconstructing the imaged target.

In an example, once the system has the series of images, the system knows the spectra. The pixels in the color standards 502a, 502b correspond to this spectra graphs 510, and after that the protocol finds a transformation matrix. The system determines a transformation matrix visually for every three images acquires, the protocol multiplies the image pixel wavelength data by the transformation matrix to obtain a hyperspectral cube. The hyperspectral cube includes the X-Y, and the third dimension is the wavelength. Therefore, for every pixel the protocol obtains a spectra visually available from the transforming.

Stage 1 can utilize a pseudo-inverse method to reconstruct a hyperspectral cube from the digital images. In Stage 1, a CMOS camera is used to capture images of the ColorChecker® standard (502a, 502b; X-Rite Passport Model #MSCCP, USA). A transformation matrix T is constructed by a generalized pseudoinverse method based on singular value decomposition (SVD) where:

T=R×PINV(D)

T=RD+(least-squares solution for RD−T)

T=RD+=R(DTD)−1DT.

Where the matrix R contains spectral reflectance factors of the calibration samples, PINV(D) is the pseudo inverse function, and the matrix D are the corresponding camera signals of the calibration samples.

Then, the predicted spectral reflectance factor R can be calculated using matrix multiplication for both the calibration (Stage 1) and verification (Stage 2) targets (discussed below).

R=T×D.

This approach for Stage 2 may have an advantage that the camera spectral sensitivity does not need to be known a priori.

The transformation matrix is the part where it forms at least one spectrum for each pixel using the detected intensity.

Stage 2 shows that a target object, shown as a hand, is imaged with low quality imaging 514a (or an average of the signals) and/or high quality imaging 514b in an illumination sequence of three illuminations and imaging. The protocol can average the signals to eventually increase the signal to noise ratio of the data. The protocol can again include acquire one image with two LEDs illuminating the target, then acquire the second image with two LEDs (e.g., different combination of LEDs), and then the third image with the third illumination pattern of LEDs such as white LEDs. Then the protocol multiplies these three images as a matrix that is multiplied by the transformation matrix that was previously obtained to generate the multispectral reflectance data cube. This operation is repeated for every image desired to be transformed into hyperspectral data cube.

It should be recognized that some of the subject matter of FIG. 5 maps to Stage 2 of FIG. 6.

The Fourier Transformation is performed after the spectrum formation as per FIG. 5. The hyperspectral decomposition systems and methods disclosed in the following publications may be used: F. Cutrale, V. Trivedi, L. A. Trinh, C. L. Chiu, J. M. Choi, M. S. Artiga, S. E. Fraser, “Hyperspectral phasor analysis enables multiplexed 5D in vivo imaging,” Nature Methods 14, 149-152 (2017); and W. Shi, E. S. Koo, M. Kitano, H. J. Chiang, L. A. Trinh, G. Turcatel, B. Steventon, C. Arnesano, D. Warburton, S. E. Fraser, F. Cutrale, “Pre-processing visualization of hyperspectral fluorescent data with Spectrally Encoded Enhanced Representations” Nature Communications 11, 726 (2020). The contents of these publications are incorporated herein in their entirety. The hyperspectral data may be quickly analyzed via the G-S plots of the Fourier coefficients of the normalized spectra, by using the following equations:

$$z ( n ) = G ( n ) + iS ( n ) \\ G ( n ) = \frac{{Σ}_{{λ}_{s}}^{{λ}_{f}} I ( λ ) cos ( n λω ) Δλ}{{Σ}_{{λ}_{s}}^{{λ}_{f}} I ( λ ) Δλ} \\ S ( n ) = \frac{{Σ}_{{λ}_{s}}^{{λ}_{f}} I ( λ ) sin ( n λω ) Δλ}{{Σ}_{{λ}_{s}}^{{λ}_{f}} I ( λ ) Δλ}$$

Where λs and λf are the starting and ending wavelengths of bands of interest, respectively; I is the intensity, ω=2π/τs, where τs is the number of spectral channels (32 in our case) and n is the harmonic (usually chosen to be either n=1 or 2, consistently).

FIG. 6 provides a schematic illustration of a two stage “pseudo-inverse” method used to reconstruct a multispectral reflectance data-cube from a series of camera images. In Stage 1, a color standard is imaged under a sequence of different lighting conditions in order to obtain their spectral reflectance factors, which is then used to solve for the transformation matrix T. In Stage 2, the transformation matrix T is used to recover the spectral information from the target object (e.g., human hand) under the same lighting sequence. The multispectral reflectance data cube is then generated as described.

Now, the present invention uses reflectance of light from the target object in view of the transformation matrix. The addition of reflected light is a different type of signal, which now can be used in hyperspectral systems for generating the multispectral reflectance data cube. For example, the protocol obtains the multispectral reflectance data cube by forming at least one spectrum for each pixel using the detected intensity and wavelength of each target wave (“intensity spectrum”) to generate the multispectral reflectance data cube. Accordingly, in FIG. 5 the step of spectrum formation 402 provides the multispectral reflectance data cube.

Then, the data processing in FIG. 5 operates from the multispectral reflectance data cube, such as by performing the Fourier Transformation 403. The processing allows for data extraction in real time.

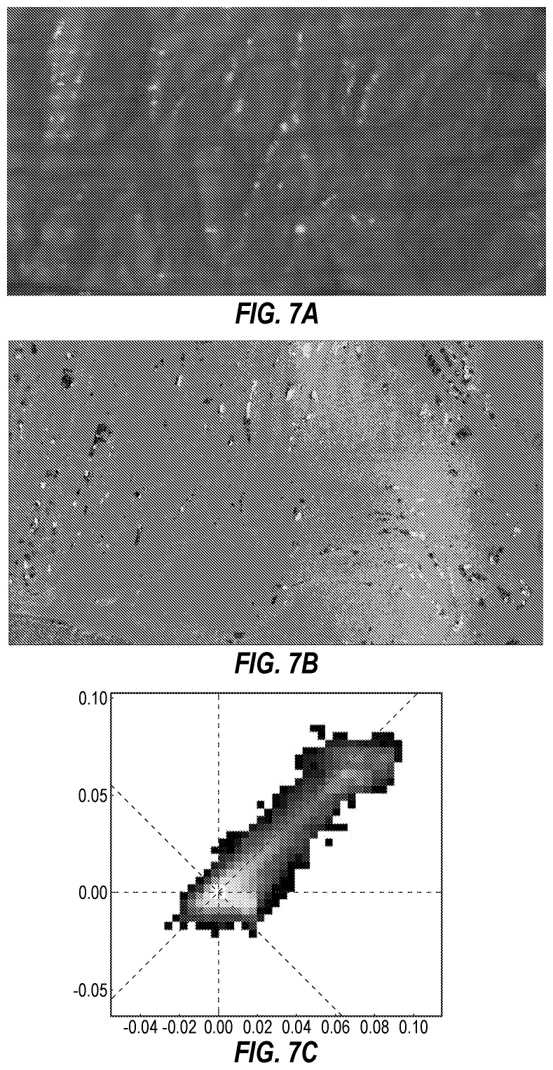

FIGS. 7A-7C show an example with an esophagus. FIG. 7A shows the esophagus under normal white light illumination (e.g., representation of the multispectral reflectance data cube). FIG. 7B shows the esophagus in a false-color hyperspectral phasor image. FIG. 7C shows the corresponding G-S histogram (e.g., phasor plot). Once the multispectral reflectance data cube from Stage 2 is obtained, the protocol can create a real and imaginary component. This protocol transforms the formed intensity spectrum (e.g., multispectral reflectance data cube) of each pixel using the Fourier Transform into a complex-valued function based on the intensity spectrum of each pixel, wherein each complex-valued function has at least one real component 404 and at least one imaginary component 405 (e.g., see FIG. 5). These are basically the real and imaginary images, which are then put into a histogram. Then, a number or an encoding is obtained for the multispectral reflectance data cube. The process performs an encoding of the spectrum signal using an harmonic and Fourier transform. In this example, the protocol uses the second harmonic and so we get two values—one real and one imaginary at one specific harmonic. Then, the protocol creates a histogram as in FIG. 7C. The protocol applies a denoising filter on both the real component and the imaginary component of each complex-valued function at least once so as to produce a denoised real value and a denoised imaginary value for each pixel.

Then the protocol forms one point on a phasor plane (“phasor point”) for each pixel by plotting the denoised real value against the denoised imaginary value of each pixel, and maps back the phasor point to a corresponding pixel on the target image based on the phasor point's geometric position on the phasor plane. The protocol assigns an arbitrary color to the corresponding pixel based on the geometric position of the phasor point on the phasor plane; and generates an unmixed color image of the target based on the assigned arbitrary color, which unmixed color image is FIG. 7B. Once the unmixed color image is obtained, then the protocol displays the unmixed color image of the target on the image forming system's display.

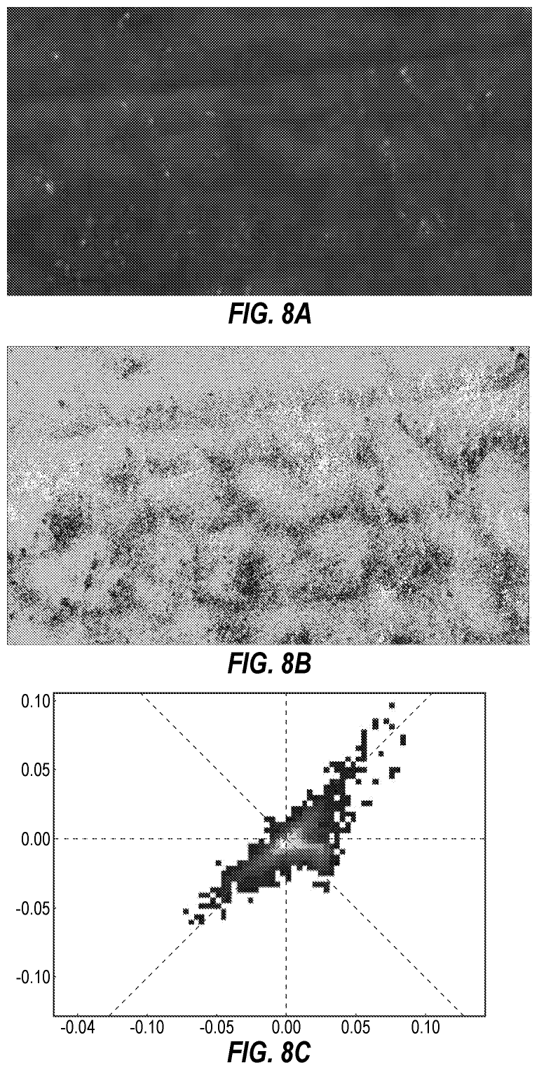

FIGS. 8A-8C show an example with a small intestine mimic. FIG. 8A shows the small intestine under normal white light illumination (e.g., representation of the multispectral reflectance data cube). FIG. 8B shows a representation in a false-color hyperspectral phasor image. FIG. 8C shows a corresponding G-S histogram (e.g., phasor plot). FIG. 8B is obtained by the processing as described in connection to FIG. 7B.

As shown in FIGS. 7A-C and 8A-C, the tissue mimics for the esophagus and small intestine visibly look similar and any discolorations may be attributed by an untrained medical assistant to shadows or non-uniformities in the illumination. However, when the images are hyperspectrally processed and graphed on in a G-S chart, the spectral distributions were distinctly different. Hence, this difference can be programmed into an automated wavelength recognition software algorithm (e.g. looking for spectrally well-defined changes in “color”) to quickly identify regions of the esophagus where dysplasia is occurring. Hence this would facilitate rapid screening and early detection of Barrett's esophagus. The importance of detection of Barrett's esophagus in this case, is that it is an early indicator of risk for esophageal adenocarcinoma.

With reference to FIG. 5, it should be recognized that the steps may be performed in different orders. For example, the denoising filer 406 can be applied between 401 and 402 or between 402 and 403. As such, FIG. 5 can be modified accordingly.

In some embodiments, the hyperspectral processing system has a configuration that: transforms the formed intensity spectrum of each pixel using a Fourier transform into a complex-valued function based on the intensity spectrum of each pixel, wherein each complex-valued function has at least one real component and at least one imaginary component; forms one phasor point on a phasor plane for each pixel by plotting the real value against the imaginary value of each pixel; maps back the phasor point to a corresponding pixel on the target image based on the phasor point's geometric position on the phasor plane; assigns an arbitrary color to the corresponding pixel based on the geometric position of the phasor point on the phasor plane; and generates an unmixed color image of the target based on the assigned arbitrary color.

In some embodiments, the hyperspectral processing system has a configuration that further includes at least one of: applying a denoising filter on both the real component and the imaginary component of each complex-valued function at least once so as to produce a denoised real value and a denoised imaginary value for each pixel, wherein the denoised real value and a denoised imaginary value are used to form the one phasor point on the phasor plane for each pixel; applying a denoising filter to the target image before forming the intensity spectrum; or applying a denoising filter before the formed intensity spectrum of each pixel is transformed.

In some embodiments, the hyperspectral processing system has a configuration that: forms a target image of the target using the detected target electromagnetic radiation, wherein the target image comprises at least two pixels, and wherein each pixel corresponds to one physical point on the target;
uses a Fourier transform to generate a complex-valued function, wherein each complex-valued function has at least one real component and at least one imaginary component; forms one phasor point on a phasor plane for each pixel by plotting the real value against the imaginary value of each pixel; maps back the phasor point to a corresponding pixel on the target image based on the phasor point's geometric position on the phasor plane; assigns an arbitrary color to the corresponding pixel based on the geometric position of the phasor point on the phasor plane; and generates an unmixed color image of the target based on the assigned arbitrary color.

In some embodiments, the methods can include using a machine learning protocol to obtain a multispectral reflectance data cube.

The system may, for example, be used in the following manner. A patient may sit in a chair during their annual health checkup. A medical assistant or nurse may spray a local analgesic into the back of the throat of the patient in order to suppress the gag reflex and to minimize discomfort to the patient. The tethered capsule can be administered with a coiled tether linked to the capsule that may be swallowed by the patient with a sip of water. The water may be mixed with a common digestible surfactant in order to minimize bubble formation in the esophagus. Gravity may uncoil the tether and the capsule may reach the gastroesophageal (GE) sphincter within 3 seconds to 5 seconds. The medical assistant may manually begin to retract the capsule from the GE sphincter at the top of the stomach and may watch on an external display screen (e.g., LCD) that may display the real-time images of the tissues in the throat from the capsule. If the medical assistant notices any unusual formations in the lining of the esophagus, the medical assistant may annotate it on the video with respect to distance markings on the tether. Depending on the interchangeable lens configuration, a low magnification lens with a wider field of view (FOV) may clearly show changes to the esophagus associated with gastroesophageal reflux disease (GERD) and Barrett's Esophagus. Also, when using a specialized lens with a larger FOV or higher magnification, the medical assistant may manually rotate and position the capsule closer to the walls of the esophagus in order to examine suspected areas of early EC. The non-circular shape of the tether can be used for the rotations. For example, a square tether cross-sectional profile can be rotated 90 degrees for each side of the tether. Depending on the number of lenses and imaging sensors, multiple views can be acquired in parallel: front, sides, and/or rear view, clearly showing the tissue, such that changes to esophagus associated with gastroesophageal reflux disease (GERD) and Barrett's Esophagus may be visualized. This entire screening process may take less than five minutes per patient. After withdrawal of the capsule via the tether, the recorded HD video images may either be reviewed by a specialist doctor or by automated machine vision software utilizing hyperspectral analysis methods for detailed analysis of each video frame.

In another example, a drone can fly over a natural environment and the lower the capsule for imaging and hyperspectral processing as described herein. A ground vehicle can travel through a small pathway to reach an area where people cannot fit, and then the imaging and hyperspectral processing can be performed. This may be useful in exploring tombs or other manmade buildings as well as natural caves. A micro-scale crane can be affixed to a well to lower the capsule via the tether, and then image the walls, bottom, or other objects or contents of the well.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

FIG. 9 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

References: PCT/US 2015/025468; F. Cutrale, V. Trivedi, L. A. Trinh, C. L. Chiu, J. M. Choi, M. S. Artiga, S. E. Fraser, “Hyperspectral phasor analysis enables multiplexed 5D in vivo imaging,” Nature Methods 14, 149-152 (2017); W. Shi, E. S. Koo, M. Kitano, H. J. Chiang, L. A. Trinh, G. Turcatel, B. Steventon, C. Arnesano, D. Warburton, S. E. Fraser, F. Cutrale, “Pre-processing visualization of hyperspectral fluorescent data with Spectrally Encoded Enhanced Representations” Nature Communications 11, 726 (2020).

1. A capsule hyperspectral system, comprising:
an imaging capsule comprising:
an illumination system having a plurality of light emitters configured for emitting a plurality of different lighting illuminations from the imaging capsule; and
a hyperspectral imaging system having at least one imaging sensor, wherein the illumination system and hyperspectral imaging system are cooperatively configured to illuminate a target with a sequence of different lighting illuminations and image the target during each of the different lighting illuminations in the sequence, and
a hyperspectral processing system having at least one processor, wherein the hyperspectral processing system is operably coupled with the hyperspectral imaging system and configured to receive images of the target therefrom and generate a multispectral reflectance data cube of the target from the received images of the target.

2. The capsule hyperspectral system of claim 1, further comprising a tether having a capsule end coupled to the imaging capsule and a system end coupled to the hyperspectral processing system.

3. The capsule hyperspectral system of claim 2, wherein the tether is communicatively coupled with the hyperspectral imaging system and hyperspectral processing system so as to pass data therebetween.

4. The capsule hyperspectral system of claim 2, further comprising a semi-rigid connection between the tether and the imaging capsule, wherein the semi-rigid connection is configured to be able to withstand manual manipulation to position the capsule without detachment of the tether from the imaging capsule.

5. The capsule hyperspectral system of claim 2, wherein the tether has a non-circular cross-sectional profile.

6. The capsule hyperspectral system of claim 2, wherein the tether comprises an external surface having markings, wherein the markings are configured to illustrate a distance from the imaging capsule when deployed.

7. The capsule hyperspectral system of claim 1, wherein the illumination system comprises at least three LEDs having at least three different color bands.

8. The capsule hyperspectral system of claim 7, wherein at least one LED is a white light LED.

9. The capsule hyperspectral system of claim 8, wherein at least two LEDs are colored LEDs with different color bands.

10. The capsule hyperspectral system of claim 7, wherein the illumination system comprises an uniformly arranged array of a plurality of LEDs.

11. The capsule hyperspectral system of claim 10, wherein the illumination system comprises at least six LEDs that include at least two white light LEDs and at least four colored LEDs with at least two different color bands.

12. The capsule hyperspectral system of claim 7, wherein an emission wavelength of each LED is selected such that a white and/or pinkish surface on healthy tissue and a red surface on non-healthy tissue can be visible identified and distinguished from each other.

13. The capsule hyperspectral system of claim 1, wherein the at least one imaging sensor and plurality of light emitters are arranged on a plate and oriented in a same direction.

14. The capsule hyperspectral system of claim 1, wherein the hyperspectral imaging system further comprises a lens system, which is a fixed lens system, detachable lens system, replaceable lens system or an interchangeable lens system.

15. The capsule hyperspectral system of claim 14, wherein the lens system has at least one lens with a field of view (FOV) in a range of at least about 90 degrees and less than about 360 degrees.

16. The capsule hyperspectral system of claim 14, wherein the lens system has at least one lens with a field of view (FOV) in a range of about 120 degree to about 180 degree.

17. The capsule hyperspectral system of claim 14, wherein the hyperspectral imaging system comprises an optical lens, an optical filter, a dispersive optic system, or a combination thereof.

18. The capsule hyperspectral system of claim 14, wherein the hyperspectral imaging system comprises a first optical lens, a second optical lens, and a dichroic mirror/beam splitter.

19. The capsule hyperspectral system of claim 14, wherein the hyperspectral imaging system comprises an optical lens, a dispersive optic, and wherein the at least one imaging sensor is an optical detector array.

20. The capsule hyperspectral system of claim 1, wherein the at least one imaging sensor is positioned in an off-centered position with respect to a central axis of the imaging capsule.

21-84. (canceled)
